(19) United States
(12) Patent Application Publication
(10) Pub. No.: US 2005/0003430 A1
(43) Pub. Date: Jan. 6, 2005

(54) Detecting polymers and polymer fragments

(76) Inventors: Donald P. Naki, San Diego, CA (US); Ayrookaran J. Poulose, Belmont, CA (US)

Correspondence Address: Genencor International, Inc., 925 Page Mill Road, Palo Alto, CA, 94034-1013, US

(21) Appl. No.: 10/893,452

(22) Filed: Jul. 16, 2004

Related U.S. Application Data
(63) Continuation of application No. 09/932,279, filed on Aug. 17, 2001.

(51) Int. Cl. C12Q 1/68; G01N 33/53; G06F 19/00; G01N 33/48; G01N 33/50

(52) U.S. Cl. 435/6; 435/7.1; 702/20

(57) ABSTRACT

An approach for detecting polymers and polymer fragments by analyzing mass analysis data of mixtures that include labeled versions of the polymers is disclosed. A library of polymer fragments is generated based on the possible fragments of a parent polymer. For each fragment in the library, a theoretical mass for both a natural version and a labeled version is generated. The labeled version may be based on a heavier isotope of an element. Data from a mass analysis, such as a mass spectrographic analysis, is received and automatically analyzed to identify whether a mass doublet is observed for each fragment in the library. The mass doublets correspond to the mass peaks of the natural and labeled versions of the fragments in the library. A determination is made whether a particular mass peak is from a labeled parent polymer or whether the particular mass peak is from an unlabeled source.

### RELATED APPLICATION

This application claims domestic priority from prior U.S. provisional application Ser. No. 60/228,198 filed Aug. 25, 2000, the entire disclosure of which is hereby incorporated by reference for all purposes as if fully set forth herein.

This Application is related to concurrently filed application with attorney docket number GC626-2, filed Aug. 17, 2001, all of which are incorporated by reference for all purposes in their entirety.

### FIELD OF THE INVENTION

The present invention relates to the analysis of polymers in mixtures, and more specifically, to detecting polymers and polymer fragments by analyzing mass data of mixtures that include labeled versions of the polymers.

### BACKGROUND OF THE INVENTION

The detection of polymers and fragments of the polymers in mixtures is a complex task. The polymer of interest is often one of many polymers in a complex mixture. Further, the polymer of interest is often broken down into smaller pieces, herein referred to as fragments. Experimenters often wish to be able to determine which fragments are observed, meaning that the experiments want to identify the fragments that are derived from the parent polymer of interest. For example, proteins may be cleaved by enzymes to produce peptides and deoxyribonucleic acid (DNA), and ribonucleic acid (RNS) may be broken into constituent nucleic acids. However, the identification of the fragments is often complicated by other polymers in the mixture breaking down into the same or similar fragments.

Furthermore, the number of potential fragments of a particular parent polymer may be so numerous as to make detecting impractical using traditional approaches that include the use of chromatography and mass spectroscopy. For example, a protein may include several hundred amino acids, and when the protein is cleaved, there may be hundreds or thousands of possible peptides produced. Two-dimensional chromatographs may be used to attempt to identify some of the peptides, but such techniques are resource intensive when trying to identify even a small number of peptides. Mass spectroscopy may be used with chromatography to determine the abundance of peptides as a function of their mass, but in a complex mixture, several proteins may be cleaved and produce the same peptides, thereby making it difficult to determine whether a particular peptide is from the protein of interest or another protein.

Based on the foregoing, it is desirable to provide improved techniques for detecting polymers and polymer fragments in mixtures. It is also desirable to have improved techniques for identifying which polymer fragments of a parent polymer are present from a large number of possible polymer fragments.

### SUMMARY OF THE INVENTION

Techniques are provided for detecting polymers and polymer fragments by analyzing mass analysis data of mixtures that include labeled versions of the polymers. According to one aspect, a method for detecting a polymer in a mixture is described. A mass based on a version of the polymer that includes a particular isotope of an element is generated, and another mass based on another version of the polymer that includes another particular isotope of the element is generated. Data based on a mass analysis of the mixture is received. A determination is made whether the data indicates an occurrence of a mass doublet that is associated with both the first mass and the second mass. If a mass doublet is identified, the corresponding polymer is likely to have been derived from a labeled parent polymer. If only a first mass is observed (i.e., a mass doublet does not occur), then the corresponding polymer is not likely to have been derived from the labeled parent polymer.

According to another aspect, a method for identifying a polymer in a mixture is described. Length values are received for fragments of the polymer. Based on the length values, a library of possible fragments of the polymer is generated for fragments having lengths consistent with the length values. For each fragment in the library, a determination is made whether the fragment is present in the mixture based on a mass spectrographic analysis of the mixture. For example, the data from a mass analysis may be analyzed to determine whether mass doublets are observed for the fragments in the library.

According to another aspect, the identification of an occurrence of a mass doublet may be based on analyzing data from a mass spectrograph for a set of scans of a chromatogram. For each scan, a search is made for a particular mass doublet. Whether or not the particular mass doublet is identified may depend on a set of factors. For example, one factor may be that there is an abundance of material corresponding to the masses of both the natural and labeled versions of the polymer or polymer fragment. Another factor may be that the abundances of the natural and labeled versions exceed a threshold abundance. Yet another factor may be determining the ratio of the natural and labeled abundances and then checking to see if the ratio thus determined is consistent with a specified ratio. For each mass doublet that is identified, a scan score is generated. If a sufficient number of consecutive scans have scan scores determined for a potential mass doublet, then a fragment score for the fragment corresponding to the mass doublet is generated. After analyzing the date to identify all potential fragments from the library, the identified fragments may be ranked based on the fragment scores.

According to other aspects, additional methods, apparatuses, and computer-readable media that implement the approaches above are described.

### BRIEF DESCRIPTION OF THE DRAWINGS

The present invention is depicted by way of example, and not by way of limitation, in the figures of the accompanying drawings and in which like reference numerals refer to similar elements and in which:

FIG. 1 is a flow diagram that depicts an approach for detecting biopolymer fragments, according to an embodiment of the invention;

FIG. 2 is a diagram that depicts an example of a chromatogram of abundance versus time;

FIG. 3 is a diagram that depicts an example of a total ion chromatogram;

FIGS. 4A-4E are a set of diagrams depicting a series of total ion chromatograms of a particular mass peak for five consecutive scans of a chromatogram;

FIG. 5 is a diagram that depicts an example of a mass doublet, according to an embodiment of the invention;

FIG. 6 is a flow diagram that depicts an approach for detecting mass doublets, according to an embodiment of the invention; and

FIG. 7 is a block diagram that depicts a computer system upon which embodiments of the invention may be implemented.

### DETAILED DESCRIPTION OF THE INVENTION

A method and apparatus for detecting polymers and polymer fragments by analyzing mass analysis data of mixtures that include labeled versions of the polymers is described. In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are depicted in block diagram form in order to avoid unnecessarily obscuring the present invention.

In the following description, the various functions shall be discussed under topic headings that appear in the following order:
I. OVERVIEW
II. CHROMATOGRAPHY AND MASS SPECTROSCOPY
III. USING LABELED VERSIONS OF POLYMERS TO PRODUCE MASS DOUBLETS
IV. AUTOMATICALLY CREATING A LIBRARY OF POLYMERS
V. AUTOMATICALLY DETECTING MASS DOUBLETS
VI. HARDWARE OVERVIEW
VII. EXTENSIONS AND ALTERNATIVES

### I. Overview

Techniques are provided for detecting polymers and polymer fragments by analyzing mass analysis data of mixtures that include labeled versions of the polymers to identify mass doublets. According to one embodiment, a natural version and a labeled version of a polymer are included in a mixture, a mass spectrographic analysis of the mixture is performed, and the resulting data is analyzed to determine the presence of mass doublets that correspond to the natural and labeled versions of the polymer.

The natural and labeled versions of the polymer have different masses because the natural version is based on the natural abundances of the isotopes of a particular element, whereas the labeled version is based on altered abundances of the isotopes of the particular element. For example, the particular element may be nitrogen so that the natural version of the polymer is mostly based on the nitrogen-14 isotope, which is the most common naturally occurring isotope of nitrogen. The labeled version of the polymer is based on nitrogen that is enriched in the nitrogen-15 isotope, resulting in a slightly heavier version of the polymer.

A mass spectrographic analysis of a chromatogram of a mixture containing both natural and labeled versions of the polymer will produce data showing pairs of mass peaks. One peak corresponds to the mass of the natural version and the other peak corresponds to the mass of the labeled version. The term “mass spectral doublet” or “mass doublet” is used herein to refer to the pair of mass peaks that correspond to the natural and labeled versions of a polymer. By using labeled versions of the polymer, mass peaks corresponding to the natural version can be distinguished from mass peaks resulting from other polymers.

According to one embodiment, a library of fragments for a polymer is automatically generated and the library used to determine whether the fragments are present in a mixture based on mass spectrographic analysis. For example, the polymer may be a protein that is cleaved by one or more enzymes, and the goal is to identify the resulting peptides that are observed as a result of the cleaving. Based on the amino acid sequence of the protein, the peptides that could possibly result from the protein being cleaved are determined. The library may include all possible peptides, or a subset of the possible peptides based on other parameters, such as all peptides within a specified length range, such as peptides having a length of five to fifteen amino acids. Whether each peptide in the library is present in the mixture may be determined based on a mass spectrographic analysis of the mixture. For example, if the protein of interest was present in the mixture using both natural and labeled versions, the data from the mass spectrographic analysis may be examined to identify whether there is a mass doublet for each peptide in the library.

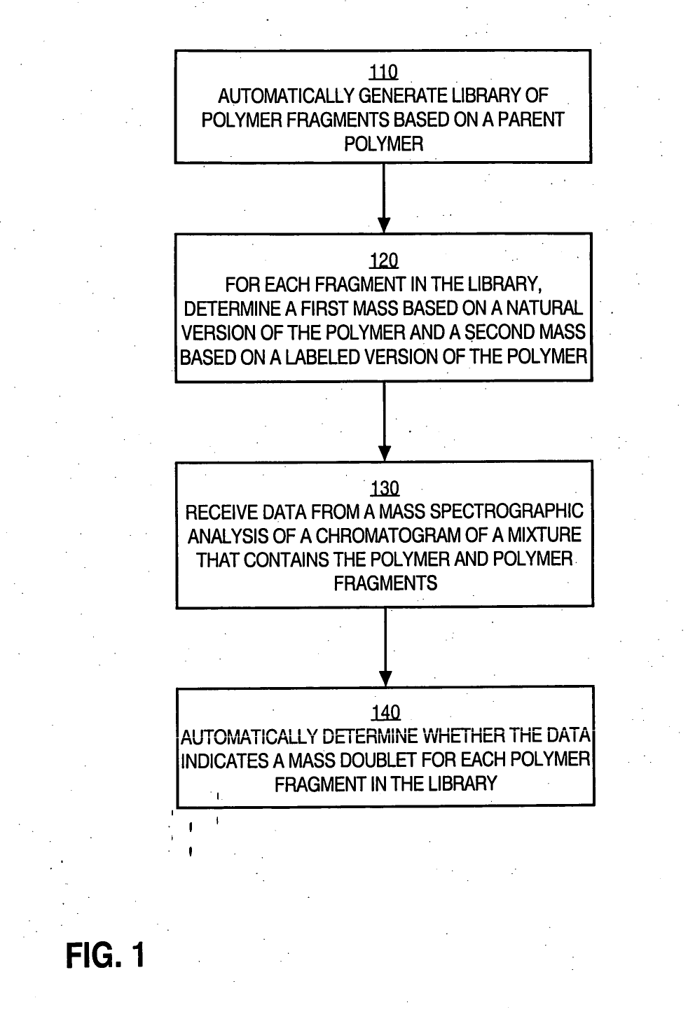

FIG. 1 is a flow diagram that depicts an approach for detecting polymer fragments, according to an embodiment of the invention. Although FIG. 1 provides a particular set of steps in a particular order, other implementations may use more or fewer steps and a different order.

In block 110, a library is automatically generated that includes polymer fragments based on a parent polymer. For example, the parent polymer may be a protein that has an amino acid sequence beginning with NGATYVEK . . . , where each letter corresponds to one of the twenty existing amino acids. A user may specify that the library include peptides having from five to seven amino acids. The library would be automatically generated by a computerized routine that determines all fragments of the parent protein that have five amino acids, such as NGATY, GATYV, etc., then those with six amino acids, such as NGATYV, etc., and then those with seven amino acids. Data identifying the peptides that are identified is stored in the automatically generated library.

In block 120, for each polymer fragment in the library, a first mass based on a natural version of the polymer fragment and a second mass based on a labeled version of the polymer fragment is determined. For example, if nitrogen is being used as the labeling element, the peptide NGATY has a first mass calculated based on nitrogen-14 as the specific isotope of nitrogen in the amino acids for the natural version of the peptide and a second mass calculated based on nitrogen-15 as the specific isotope of nitrogen in the amino acids for the labeled version of the peptide.

In block 130, data from a mass spectrographic analysis of a chromatogram of a mixture that contains the polymer and polymer fragments is received. For example, the mixture may contain the protein that begins with NGATYVEK . . . and that contains peptides of that protein, such as may result from cleaving the protein with an enzyme. The mixture is input to a chromatography column that in turn provides input to a mass spectrograph that produces a set of data describing the abundance of the detected masses for each time interval of the chromatogram.

In block 140, an automated determination is made as to whether the data from the mass spectrograph indicates a mass doublet for each polymer fragment in the library. For example, the data is automatically examined for the masses corresponding to the natural and labeled versions of the peptide NGATY to identify whether a mass doublet peak is observed. If peaks corresponding to both the natural and labeled masses for NGATY are identified, then that tends to indicate that NGATY is one peptide resulting from the cleaving of the parent protein. However, if only a peak corresponding to the mass of the natural version is observed, then that tends to indicate that NGATY is a peptide resulting from another source, such as the cleaving of another unlabeled protein in the mixture. The data from the mass spectrograph is automatically examined to look for mass doublets for each peptide in the library.

Although the discussion herein provides examples that are based on proteins and peptides, the techniques described are applicable to any type of polymer and any type of polymer fragment. For example, proteins are one example of a biological polymer, or biopolymers. Proteins are composed of a sequence of amino acids and may be cleaved into peptides that are shorter sequences of amino acids. Other examples of biopolymers include DNA and RNA that are composed of nucleotides and that can be fragmented into nucleic acids that are shorter sequences of nucleotides. Therefore, for simplicity and clarity of explanation, the examples herein focus on proteins and peptides, but the techniques are applicable to any polymers and polymer fragments.

### II. Chromatography and Mass Spectroscopy

Chromatography is used to separate the constituents of a mixture based on one or more properties for the particular chromatography technique. A sample of the mixture is placed in the top of a chromatography column that contains a chromatographic medium, or matrix, that is capable of fractionating the mixture. Examples of chromatographic techniques that may be used include, but are not limited to, the following: reverse phase chromatography, anion or cation exchange chromatography, open-column chromatography, high-pressure liquid chromatography (HPLC), and reverse-phase HPLC. Other separation techniques that may be used include, but are not limited to, the following: capillary electrophoresis and column chromatography that employs the combination of successive chromatographic techniques, such as ion exchange and reverse-phase chromatography. Also, precipitation and ultrafiltration may be used as initial clean-up steps as part of the peptide separation protocol.

The different constituents of the mixture fall through the matrix of the column at different rates depending on each constituent's properties, thereby separating the constituents. The output of the chromatography process is a chromatogram showing the abundance of the constituents that are leaving, or “eluting,” from the column as a function of time. While the chromatogram provides information about how much material is eluting from the bottom of the column and when the material elutes from the column, the chromatogram does not identify which polymers or polymer fragments are eluting from the column.

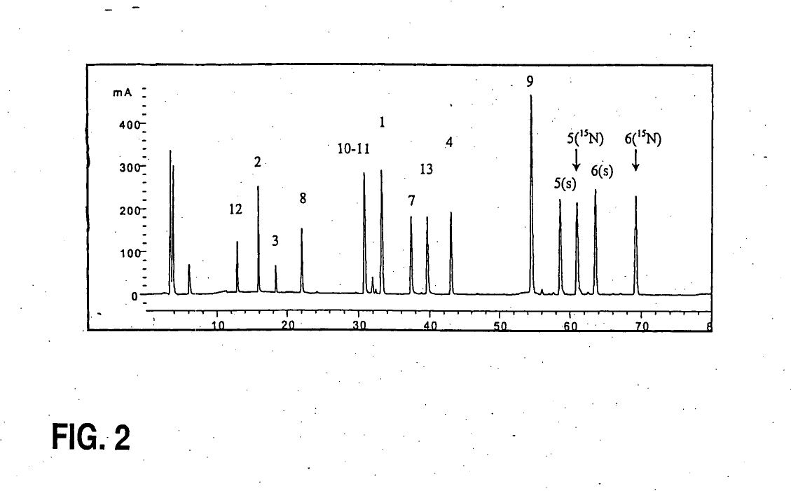

FIG. 2 is a diagram that depicts an example of a chromatogram of abundance versus time. The peaks depicted in FIG. 2 correspond to thirteen different peptides, numbered one through thirteen, that have been identified by other means. For peptides five and six, two peaks are shown, one for the natural version denoted by “s” and one for the nitrogen-15 labeled version.

The output of the chromatography column may be the input to a mass analyzer that provides mass information at a given time from the chromatogram. For the examples herein, a mass spectrometer is described. However, other mass analysis devices that work off of other properties, such as differing electromagnetic wavelengths, may also be used.

With a mass spectrometer, the material is ionized to determine the materials' mass. For example, the material may be a mixture of polymers. Each polymer may be ionized into one of a number of charge states, such as singly ionized, doubly ionized, etc. Some mass spectrometers only produce single ionized material, while others work with multiple charge states. The output of the mass spectrometer is a measurement of abundance of the material as a function of the mass/charge (m/z) state. The mass spectrometry output may be referred to as a total ion chromatogram.

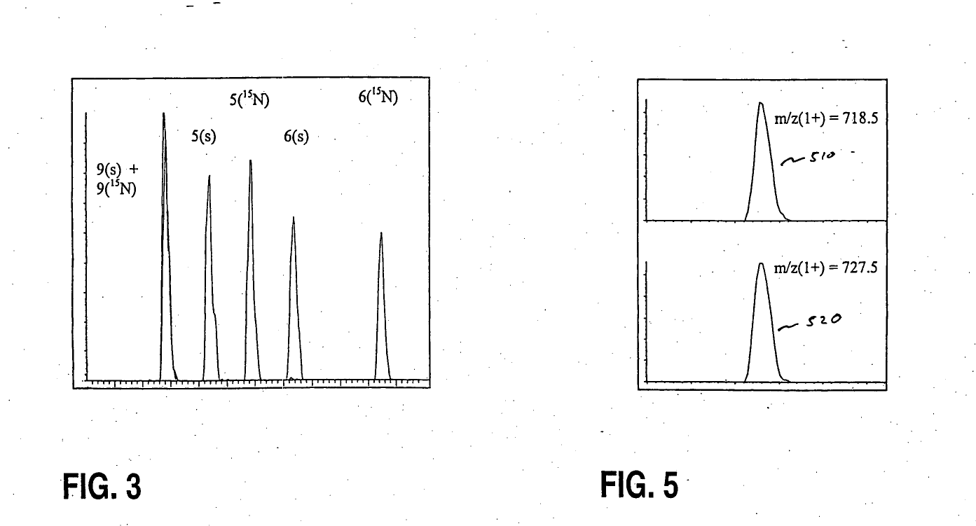

FIG. 3 is a diagram that depicts an example of a total ion chromatogram. The peaks shown in FIG. 3 correspond to the peaks for peptides five, six, and nine in FIG. 2. For each of the three peptides, two peaks are shown, one for the natural version denoted by “s” and one for the nitrogen-15 labeled version.

The mass spectrometer functions by analyzing the output of the chromatography column in time slices, or “scans.” For example, the chromatogram may contain data for hundreds of seconds of output, and the mass spectrometer analyzes the output of the chromatography column in one-second increments. Each total ion chromatogram from the mass spectrometer shows how much material is present during the scan of the chromatography output as a function of the mass/charge of the material present in the scan. Each scan may be filtered to only look at one or more masses (or ranges of masses). By filtering and then combining the mass spectrometry results for each scan, the abundance for a particular mass may be determined as a function of time.

Any suitable mass spectrometry device may be used, including but not limited to, the following: an electrospray ionization (ESI) single or triple-quadropule mass spectrometer, an ion-trap ESI mass spectrometer, Fourier-transform ion cyclotron resonance mass spectrometer, a MALDI time-of-flight mass spectrometer, a quadrupole ion trap mass spectrometer, or any other mass spectrometer having any combination of suitable source and detector.

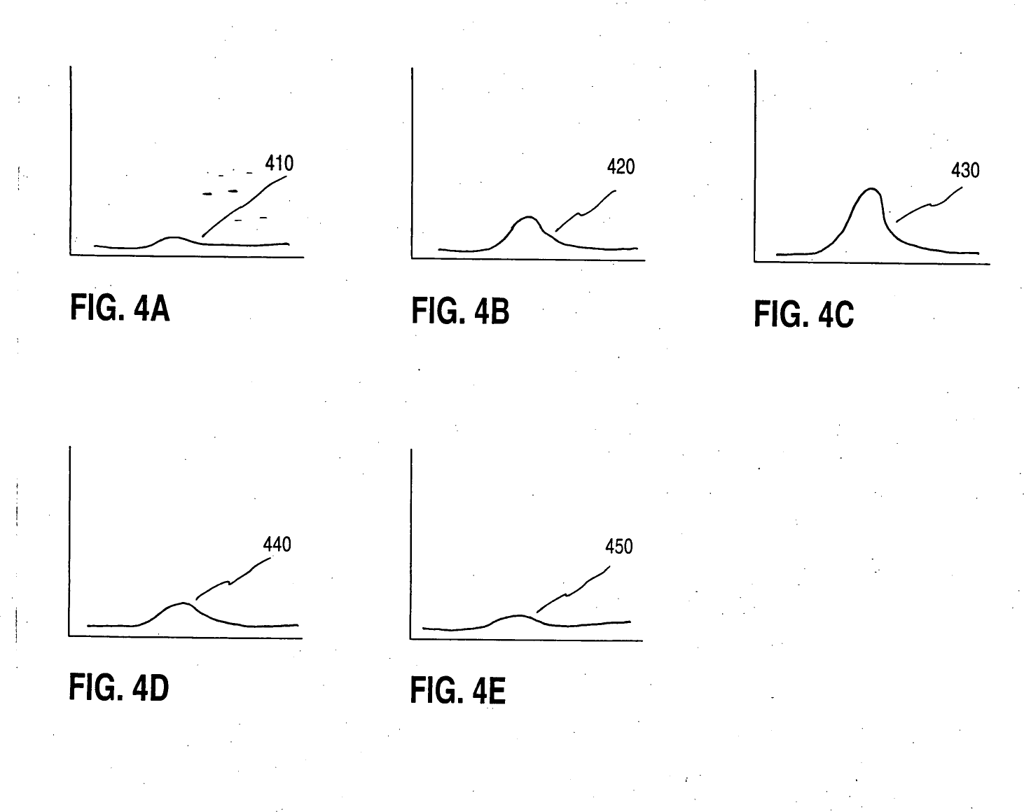

FIGS. 4A-4E are a set of diagrams depicting a series of total ion chromatograms of a particular mass peak for five consecutive scans of a chromatogram. Assume that each scan has a duration of one second, that only one polymer is present, and that the chromatography column uses the molecular weight as the property to separate the mixture into the constituents. The polymer does not elute from the chromatography column all at once. Rather, the polymer starts to slowly elute and then builds up to a peak that then tapers off. Thus, the polymer may elute over a particular time period that is typically longer than the duration of a single scan by the mass spectrograph. For this example, the polymer is assumed to elute over a time period of five seconds, which is covered by five one-second scans.

In FIG. 4A, the total ion chromatogram depicts a peak 410 that is very small for the first scan for the time period of zero to one second of output from the chromatography column. In FIG. 4B, a peak 420, which is larger than peak 410, is depicted for the second scan for the time period of one to two seconds of output, thereby showing the increase in the elution of the material from the chromatography column. In FIG. 4C, a peak 430 is depicted that represents the abundance for the third scan. In FIG. 4D, a peak 440 is depicted that illustrates the decrease in abundance during the fourth scan as compared to the third scan. Finally, in FIG. 4E, a peak 450 depicts the abundance gradually dropping off from peaks 430 and 440.

### III. Using Labeled Versions of Polymers to Produce Mass Doublets

According to one embodiment, both a natural version and a labeled version of a polymer are used to produce mass doublets that may be observed in the output of a mass analysis. The mass doublets may correspond to one or more labeled polymers in the mixture, one or more polymer fragments of the labeled polymers, or both. For example, the polymer may be a protein that is cleaved into peptides, and mass doublets may appear for both the protein and a group of peptides cleaved from the protein. In some experiments, there is a particular protein, referred to as the “protein of interest,” that is cleaved by an enzyme, and the goal is to identify the peptides that appear, or are “observed,” from the action of the enzyme.

A “labeled” version of the protein of interest may be used that is the same as the “natural” version of the protein except that the labeled version includes one or more known differences. In general, the natural and labeled versions of the protein have similar chemical and physical properties, but the two versions differ in at least one chemical or physical property. For example, one labeling approach may employ amino acid sequences that are homologous, but not identical, to each other (i.e., the labeled version has one or more amino acid substitutions, insertions, or deletions). As more specific examples, the labeled version may share at least 90, 95, or 98 percent homology with the natural version. Other approaches include, but are not limited to, tagging the labeled version to alter at least one chemical or physical property. Furthermore, the approaches herein may be combined, such as using homologous proteins with the isotope labeling that is described below.

Another example of a labeling approach is to use a different stable isotope of a particular element. For example, the element may be nitrogen, for which the most common naturally occurring isotope is nitrogen-14. The protein based on naturally occurring nitrogen is the natural version of the protein and may be referred to as the nitrogen-14 version. Another version of the protein, the labeled version, may be created based on nitrogen-15, which is the less common naturally occurring isotope of nitrogen. The natural and labeled versions of the protein are the same except that the labeled version has a slightly larger mass because the mass of nitrogen-15 is about 15 atomic mass units (amu) while the mass of nitrogen-14 is about 14 amu. Because the natural and labeled versions are very similar in mass, the two versions co-elute (i.e., the two versions elute from the chromatography column at about the same time).

While the examples herein are described in terms of nitrogen-14 and nitrogen-15 as the isotopes used for the natural and labeled versions, respectively, other elements and isotopes may be used. For example, carbon may be used with carbon-12 in the natural version and carbon-13 in the labeled version, or hydrogen-1 and hydrogen-2 may be used. Other elements may be used that include other isotopes, such as sulfur and phosphorous, and the isotopes used may include radioactive isotopes, such as phosphorous-32, in addition to stable isotopes.

When a mass spectrographic analysis is performed for a mixture that includes both natural and labeled versions of a protein of interest that is broken down into peptides, the peptides that are from the labeled protein of interest will be observed in both the natural and labeled masses as part of a mass doublet. Any peptides that are cleaved from other proteins that are not labeled are observed as single peaks that correspond to the natural versions of such peptides. Therefore, peptides from the protein of interest are identified based on the presence of mass doublets, whereas peptides from other proteins that were not labeled are observed as having only single peaks. The techniques described herein are suitable for analyzing polymers and polymer fragments that are just a small proportion of the mixture.

FIG. 5 is a diagram that depicts an example of a mass doublet for a protein that is singly charged, according to an embodiment of the invention. FIG. 5 depicts a peak 510 that corresponds to the natural version of the protein that has a mass of about 718.5 amu. FIG. 5 also depicts a peak 520 that corresponds to the nitrogen-15 labeled version of the protein that has a mass of about 727.5 amu. Because the mass doublet consisting of peaks 510 and 520 is observed, the naturally occurring peptide of mass 718.5 amu is identified as originating from the protein of interest. If only peak 510 was observed, and there was no peak corresponding to the labeled version of the protein of interest, then the peptide of mass 718.5 amu would not be identified as originating from the protein of interest.

### IV. Automatically Creating a Library of Polymers

Typically, a protein may be fragmented into a large number of peptides by an enzyme or chemical activity that is capable of cleaving the protein at particular cleavage sites. For example, a suitable fragmenting technique may include, but is not limited to, one or more of the following: the enzyme trypsin that hydrolyzes peptide bonds on the carboxyl side of lysine and arginine (with the exception of lysine or arginine followed by proline), the enzyme chymotrypsin that hydrolyzes peptide bonds preferably on the carboxyl sides of aromatic residues (i.e., phenylalanine, tyrosine, and tryptophan), and cyanogens bromide (CNBr) that chemically cleaves proteins at methionine residues.

Different fragmenting techniques may produce different sets of peptides from the same parent protein. While the protein may be known or previously identified, the peptides that result from a particular fragmenting technique may not be known. Thus, the identities of the resulting peptides may be one goal of the experiment. Because the protein may consist of several hundred amino acids, the fragmenting technique may produce any of a very large number of possible peptides, even within a relatively narrow range of peptides such as peptides having lengths of ten to fifteen amino acids. As a result, traditional approaches for identifying the peptides that result from the fragmentation are often time consuming and resource intensive due to the large number of potential peptides.

According to one embodiment, a library of polymers is automatically created for use in detecting mass doublets. For example, the amino acid sequence for a protein may be provided as input to a computerized routine and every possible peptide that may result from the sequence is identified by the routine. Data identifying the peptides is stored in the library. As another example, the experimenters may expect only peptides within a certain range of lengths to be observed, and the automatically generated library may be limited to peptides that are within the range. For example, if the range were eleven to twenty-three, then the library includes only peptides having a length of eleven to twenty-three amino acids. As additional examples, a minimum length, a maximum length, a set of ranges, one or more specified lengths, a combination of the examples herein, or any other suitable criteria may be used to specify which peptides to include in the library.

For example, the protein of interest may be described by an amino acid sequence that begins as follows: NGATYVEKTAVN . . . . The criteria for generating the peptides for the library may be that only peptides having at least a length of ten amino acids but not greater than twenty amino acids are to be included. The criteria may be provided by the experimenters to the library generating routine based on a biological rationale or previous experience. Based on the criteria, the peptides for the library are included by executing the routine to identify every subsequence of the protein that has from ten to twenty amino acids.

The library generating routine may generate the library by making one processing pass through the protein for each length in the specified range. For example, if the library is constructed starting with peptides having ten amino acids, the first peptide identified may be the peptide having the first ten amino acids in the sequence of the protein of interest (e.g., NGATYVEKTA). The next identified peptide may be the peptide defined by the second through eleventh amino acids in the sequence of the protein of interest (e.g., GATYVEKTAV). This process is repeated for all possible peptides having ten amino acids until the end of the sequence of the protein of interest is reached. The process is then repeated from the start of the sequence, for peptides having eleven amino acids, then again for those having twelve amino acids, and so on until all peptides having lengths within the specified range of ten to twenty amino acids are identified.

### V. Automatically Detecting Mass Doublets

According to one embodiment, a mass doublet is automatically detected by determining theoretical masses for the natural and labeled versions of a polymer and causing a mass doublet detecting routine to search each scan of the mass analysis data for the mass doublet. When a potential mass doublet is detected, routines perform the automated steps of generating a score for the scan and scoring the polymer if a sufficient number of consecutive scans are identified to have an occurrence of the mass doublet. Whether or not the mass doublet detection routine determines that a mass doublet is present is based on specified criteria. Examples of such criteria include, but are not limited to, the following: whether both the natural and labeled masses are present, whether both masses exceed a specified threshold, and whether the ratio of the masses are consistent with a specified ratio. According to other aspects, the detection of mass doublets may be performed for each polymer in a library, the detected mass doublets may be listed or ranked based on the scores, and the abundance of a polymer may be provided as a function of time.

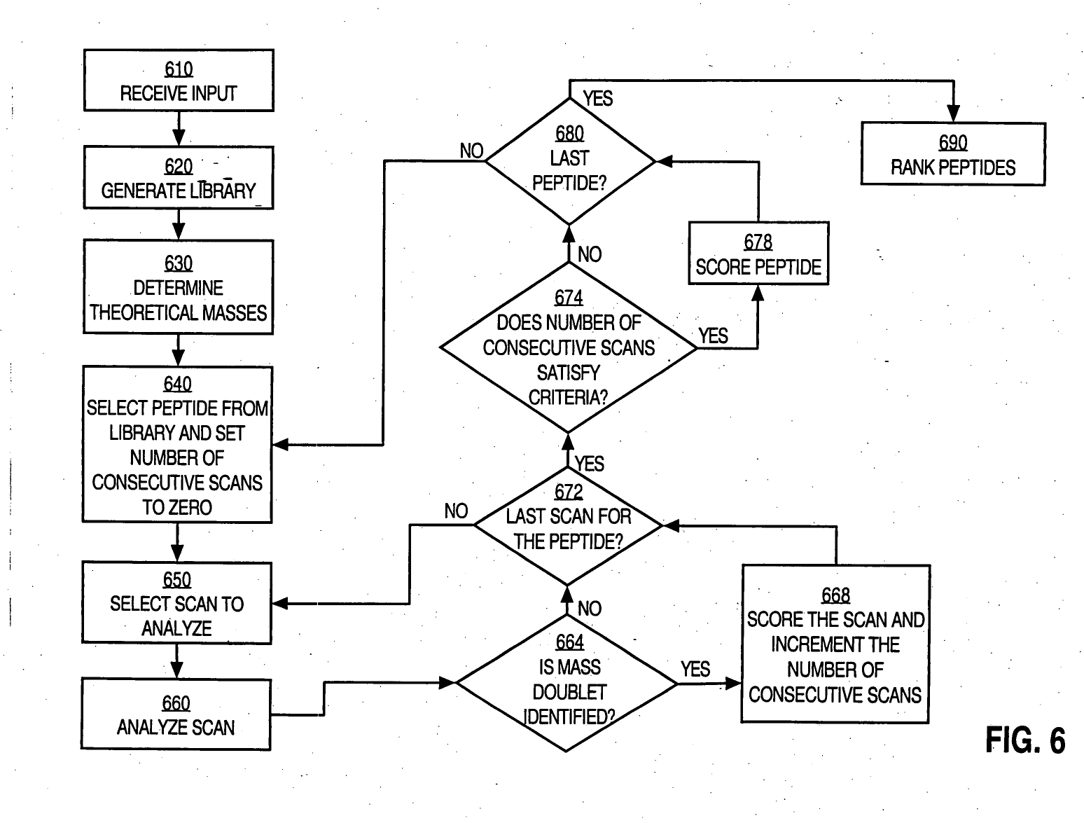

FIG. 6 is a flow diagram that depicts an approach for detecting mass doublets, according to an embodiment of the invention. Although FIG. 6 provides a particular set of steps in a particular order, other implementations may use more or fewer steps and a different order. For the purposes of simplification, the following explanation focuses on a nitrogen-15 labeled protein that is fragmented into peptides and analyzed using a mass spectrometer, although any polymer or set of polymers using other labeling isotopes or labeling approaches may be analyzed by a suitable mass analysis technique.

In block 610, input is received. The input includes mass data that describes the abundance of different masses, such as the data from a mass spectrograph of a chromatogram. The mass data typically includes data for a number of scans of a chromatogram, with each scan corresponding to a specified time interval of the chromatogram. The mass data may be stored in a file, database, or other suitable mechanism in a suitable format, such as the Finnigan LCQ QualBrowser text file format.

The input may include one or more of the following parameters that are described further below: the amino acid sequence of the protein, the minimum and maximum length of peptides expected when the protein is fragmented, the mass/charge accuracy of the mass spectrometer used for the mass analysis, an abundance threshold for detecting mass doublet peaks, an expected ratio of the natural to labeled versions of the peptides of interest, the number of consecutive scans in a candidate mass doublet must be detected before the presence of the corresponding peptide is considered to be established, the starting and ending time in the mass analysis data to search for mass doublets, and the range of the number of charge states expected for the peptides from the mass spectrometer. The input values may be supplied by a user, a stored file, an apparatus, a software program, or any other suitable source of input.

In block 620, a library is generated. The library may be referred to as a “virtual peptide library” represents all possible subsequences of the protein that satisfy specified criteria. Creation of the library is described in the previous section. The search for mass doublets in the mass spectrography data may be performed for any number or all of the peptides in the library. As an alternative, instead of generating a library in block 620, a previously generated library may be identified and retrieved.

In block 630, theoretical, or “average isotopic,” masses are determined. For example, for each peptide in the library, the theoretical mass of both the natural version based on the nitrogen-14 isotope and the labeled version based on the nitrogen-15 isotope are calculated. The theoretical mass may be calculated for more than one charge state, as determined by a specified range of charge states expected for the mass spectrograph. Because the mass spectrograph data provides abundance as a function of the mass/charge ratio, the theoretical masses for the different potential charge states may be generated as necessary.

In block 640, a peptide from the library is selected and the number of consecutive scans is set to zero. The selected peptide is the subject of the searching steps described below. The number of consecutive scans is a counter that is used as described below.

In block 650, a scan to be analyzed is selected. For example, the scan may be the first scan in the mass spectrograph data, the first scan corresponding to a specified start time, or the next scan following a previously analyzed scan.

In block 660, the scan is analyzed to determine whether a mass doublet is identified in the mass spectrograph data. The analysis may focus on one or more factors. For example, one factor may be whether the data for the scan selected in block 650 shows an abundance for the mass/charge corresponding to each of the natural and labeled theoretical masses determined in block 630 for the peptide selected in block 640. If a range of charge states were previously specified, the theoretical masses for each charge state may be checked.

Because the mass spectrograph data varies due to the uncertainty of the device, the mass/charge accuracy for the device may be used to identify whether an abundance for the theoretical masses is present. For example, the mass/charge accuracy may be expressed as a percentage, for example 0.5%, and the identification for a particular theoretical mass may include searching for abundances within 0.5% of the theoretical mass determined in block 630.

Another factor that may be used is an abundance threshold. The mass spectrograph output may reflect a variable amount of background noise that is present regardless of whether actual material of a given mass is actually present. Therefore, an abundance threshold may be specified and each potential peak that corresponds to a theoretical mass may be compared to the abundance threshold, and potential peaks that fall below the threshold are discarded from consideration.

Yet another factor that may be used is an expected ratio of the natural to labeled versions of the peptide. The experimenters often know the proportion of natural to labeled versions of the protein in the mixture based on the experimental procedure. Therefore, any peptides that are fragmented from the natural and labeled versions of the parent protein should be observed in the same ratio. Also, a specified error for the ratio may be provided, such that the mass data may be analyzed to determine if the ratio of natural to labeled versions of the peptide fall within a range based on the expected ratio and the specified error (e.g., from a minimum that is based on the expected ratio less the error to a maximum that is based on the expected ratio plus the error).

Other factors in addition to those listed above may also be used, and particular implementations may use some, all, or none of the example factors described herein.

In block 664, a determination is made as to whether a mass doublet is identified. For example, if all three of the example factors above are used, a mass doublet is identified if (1) an abundance is identified corresponding to both the natural and labeled theoretical masses, (2) the identified abundances exceed the abundance threshold, and (3) the observed ratio of natural to labeled versions of the peptide are within the range based on the expected ratio and the specified error. If all three criteria are satisfied, then an occurrence of the mass doublet is said to have been identified. Otherwise, if fewer or none of the criteria are satisfied, the mass doublet is said to not have been identified.

If in block 664 a mass doublet was not identified, the method continues to block 672.

If in block 664 a mass doublet is identified, then in block 668, the scan is scored and the number of consecutive scans is incremented. The score determined in block 668 may be referred to as a “scan score.” For example, the scan score may be determined as the sum of the average abundance of the peaks corresponding to the masses of the natural version and the labeled version of the peptide. Other scoring approaches may be used, such as assigning a specified value, summing the largest abundance values of the two peaks, or basing the scan score on only one of the two peaks. After block 668, the method proceeds to block 672.

In block 672, a determination is made whether the just analyzed scan is the last scan for the peptide. For example, there may be no more data for scans beyond the last analyzed peptide, or the last analyzed peptide may be the last scan within a specified time range to be analyzed. If the scan is not the last scan to be analyzed for the peptide, the method returns to block 650 where another scan is selected. If the scan is the last scan to be analyzed, the method continues to block 674.

In block 674, a check is made to determine if the number of consecutive scans meets or exceeds a specified number of scans. For example, the experimenters may have provided a minimum number of consecutive scans for which scores in block 668 must be generated to consider that a true mass doublet has been identified. Other criteria may be used in place of the number of consecutive scans. For example, a cumulative score from the scores generated in block 668 may be tracked and the check in block 674 may be to determine whether the cumulative score satisfies specified criteria, such as that the cumulative score meets or exceeds the specified score.

If in block 674 the number of consecutive scores is not sufficient, the method proceeds to block 680. However, if the number of consecutive scores is sufficient, then the method moves from block 674 to block 678.

In block 678, the peptide is scored. The score determined in block 678 may be referred to generally as a “fragment score” or more specifically for this protein example, as a “peptide score.” For example, the peptide score may be determined based on a sum of the scan scores that correspond to the number of consecutive scans for which scan scores were generated in block 668. The method then continues to block 680.

In block 680, a determination is made whether the selected peptide is the last peptide from the library to be analyzed. If the peptide is not the last peptide, the method returns to block 640 where another peptide is selected from the library. If the peptide is the last peptide, then the method continues to block 690.

In block 690, the peptides are ranked based on the peptide scores. For example, a listing of the peptides based on decreasing peptide scores may be generated and provided to a user. Other post processing may also be performed, such as providing plots of the abundances as a function of time for the natural and labeled versions of a particular peptide, either together, separately, or combined with any other available data.

Although the example described above with reference to FIG. 6 focused on one protein of interest, a set of proteins of interest may also be used to generate the library and for which the above steps are performed. Further, as noted above, the examples herein focus on proteins and peptides, but the techniques may be used for other biopolymers or more generally any other polymers and polymer fragments. Also, the above example used nitrogen-15 for the labeled version of the peptides, but other isotopes may be used, including but not limited to, hydrogen-2 and carbon-13.

The scan scores and peptide scores obtained from blocks 668 and 678, respectively, may be used to determine quantity measurements of the identified peptides. For example, the ranked list described above may be used to judge the abundance of a particular peptide relative to the other peptides that are identified, thereby providing a qualitative quantity measurement.

Furthermore, the approaches used to produce the scores may be chosen such that the scores provide a measure of the relative quantity measurement of the abundance of the peptides (e.g., if the score for one peptide is twice that of another peptide, then that indicates the one peptide is twice as abundant as the other peptide).

In addition, a known standard may be used to determine an absolute quantity measurement of the abundance of the peptides. For example, given a known amount of a labeled protein of interest in the mixture, the ratio of the abundance of the natural version of a peptide of the protein of interest to the abundance of the labeled version of the peptide of the protein of interest may be used to determine the absolute quantity of the natural version of the peptide.

### VI. Hardware Overview

The approach for detecting polymers and polymer fragments by analyzing mass spectrography data of mixtures that include labeled versions of the polymers to identify mass doublets described herein may be implemented in a variety of ways and the invention is not limited to any particular implementation. The approach may be integrated into a mass spectroscopy system, a mass spectroscopy device, a general purpose computer, or the approach may be implemented as a stand-alone mechanism. Furthermore, the approach may be implemented in computer software, hardware, or a combination thereof.

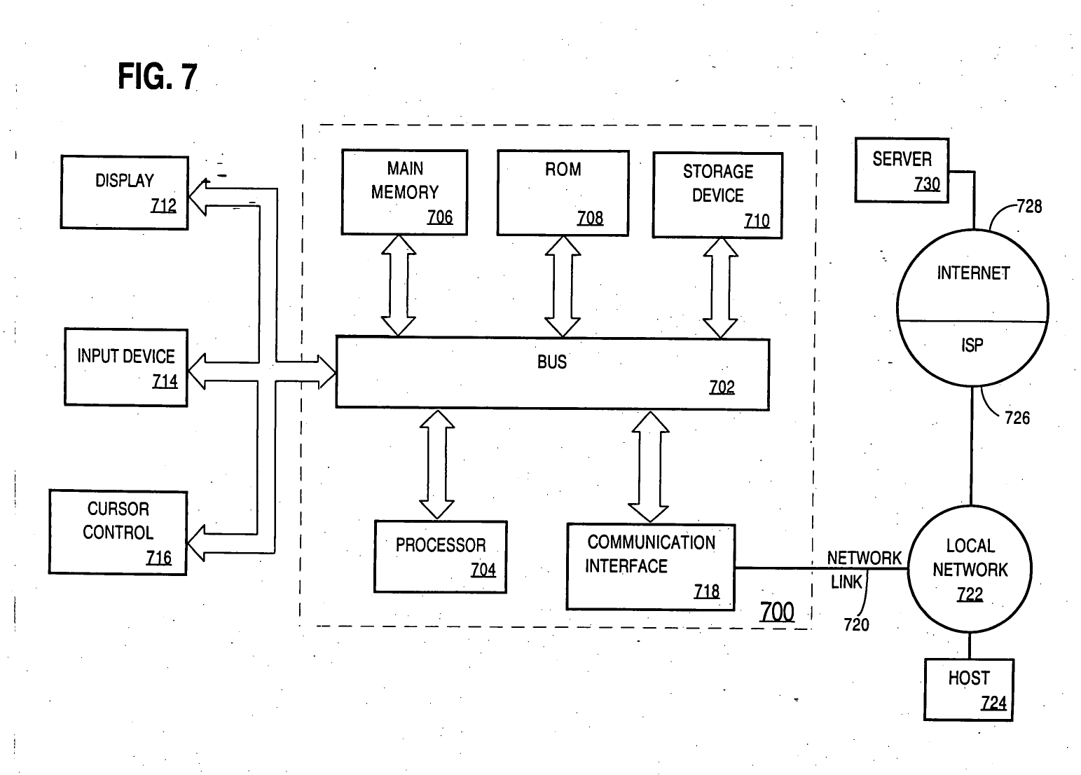

FIG. 7 is a block diagram that depicts a computer system 700 upon which an embodiment of the invention may be implemented. Computer system 700 includes a bus 702 or other communication mechanism for communicating information, and a processor 704 coupled with bus 702 for processing information. Computer system 700 also includes a main memory 706, such as a random access memory (RAM) or other dynamic storage device, coupled to bus 702 for storing information and instructions to be executed by processor 704. Main memory 706 also may be used for storing temporary variables or other intermediate information during execution of instructions to be executed by processor 704. Computer system 700 further includes a read only memory (ROM) 708 or other static storage device coupled to bus 702 for storing static information and instructions for processor 704. A storage device 710, such as a magnetic disk or optical disk, is provided and coupled to bus 702 for storing information and instructions.

Computer system 700 may be coupled via bus 702 to a display 712, such as a cathode ray tube (CRT), for displaying information to a computer user. An input device 714, including alphanumeric and other keys, is coupled to bus 702 for communicating information and command selections to processor 704. Another type of user input device is cursor control 716, such as a mouse, a trackball, or cursor direction keys for communicating direction information and command selections to processor 704 and for controlling cursor movement on display 712. This input device typically has two degrees of freedom in two axes, a first axis (e.g., x) and a second axis (e.g., y), that allows the device to specify positions in a plane.

The invention is related to the use of computer system 700 for implementing the techniques described herein. According to one embodiment of the invention, those techniques are performed by computer system 700 in response to processor 704 executing one or more sequences of one or more instructions contained in main memory 706. Such instructions may be read into main memory 706 from another computer-readable medium, such as storage device 710. Execution of the sequences of instructions contained in main memory 706 causes processor 704 to perform the process steps described herein. In alternative embodiments, hard-wired circuitry may be used in place of or in combination with software instructions to implement the invention. Thus, embodiments of the invention are not limited to any specific combination of hardware circuitry and software.

The term “computer-readable medium” as used herein refers to any medium that participates in providing instructions to processor 704 for execution. Such a medium may take many forms, including but not limited to, non-volatile media, volatile media, and transmission media. Non-volatile media includes, for example, optical or magnetic disks, such as storage device 710. Volatile media includes dynamic memory, such as main memory 706. Transmission media includes coaxial cables, copper wire and fiber optics, including the wires that comprise bus 702. Transmission media can also take the form of acoustic or light waves, such as those generated during radio-wave and infra-red data communications.

Common forms of computer-readable media include, for example, a floppy disk, a flexible disk, hard disk, magnetic tape, or any other magnetic medium, a CD-ROM, any other optical medium, punchcards, papertape, any other physical medium with patterns of holes, a RAM, a PROM, and EPROM, a FLASH-EPROM, any other memory chip or cartridge, a carrier wave as described hereinafter, or any other medium from which a computer can read.

Various forms of computer readable media may be involved in carrying one or more sequences of one or more instructions to processor 704 for execution. For example, the instructions may initially be carried on a magnetic disk of a remote computer. The remote computer can load the instructions into its dynamic memory and send the instructions over a telephone line using a modem. A modem local to computer system 700 can receive the data on the telephone line and use an infra-red transmitter to convert the data to an infra-red signal. An infra-red detector can receive the data carried in the infra-red signal and appropriate circuitry can place the data on bus 702. Bus 702 carries the data to main memory 706, from which processor 704 retrieves and executes the instructions. The instructions received by main memory 706 may optionally be stored on storage device 710 either before or after execution by processor 704.

Computer system 700 also includes a communication interface 718 coupled to bus 702. Communication interface 718 provides a two-way data communication coupling to a network link 720 that is connected to a local network 722. For example, communication interface 718 may be an integrated services digital network (ISDN) card or a modem to provide a data communication connection to a corresponding type of telephone line. As another example, communication interface 718 may be a local area network (LAN) card to provide a data communication connection to a compatible LAN. Wireless links may also be implemented. In any such implementation, communication interface 718 sends and receives electrical, electromagnetic or optical signals that carry digital data streams representing various types of information.

Network link 720 typically provides data communication through one or more networks to other data devices. For example, network link 720 may provide a connection through local network 722 to a host computer 724 or to data equipment operated by an Internet Service Provider (ISP) 726. ISP 726 in turn provides data communication services through the world wide packet data communication network now commonly referred to as the “Internet” 728. Local network 722 and Internet 728 both use electrical, electromagnetic or optical signals that carry digital data streams. The signals through the various networks and the signals on network link 720 and through communication interface 718, which carry the digital data to and from computer system 700, are exemplary forms of carrier waves transporting the information.

Computer system 700 can send messages and receive data, including program code, through the network(s), network link 720 and communication interface 718. In the Internet example, a server 730 might transmit a requested code for an application program through Internet 728, ISP 726, local network 722 and communication interface 718.

The received code may be executed by processor 704 as it is received, and/or stored in storage device 710, or other non-volatile storage for later execution. In this manner, computer system 700 may obtain application code in the form of a carrier wave.

### VII. Extensions and Alternatives

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense.

1. A method for detecting a polymer in a mixture, the method comprising the computer-implemented steps of:
generating both a first mass based on a first version of the polymer that includes a first isotope of an element and a second mass based on a second version of the polymer that includes a second isotope of the element;
receiving data based on a mass analysis of the mixture; and
determining whether the data indicates an occurrence of a mass doublet that is associated with both the first mass and the second mass.

2. The method of claim 1, wherein the polymer is a biopolymer.

3. The method of claim 2, wherein the biopolymer is comprised of one or more amino acids.

4. The method of claim 2, wherein the biopolymer is comprised of one or more nucleotides.

5. The method of claim 1, wherein the mass analysis is a mass spectrographic analysis.

6. The method of claim 1, wherein the polymer is a particular polymer of a plurality of polymers, and wherein the method further comprises the computer-implemented steps of:
for each polymer of the plurality of polymers, performing the steps of generating and determining.

7. The method of claim 6, wherein the plurality of polymers is identified in a library, and wherein the method further comprises the computer-implemented steps of:
receiving one or more length values; and
based on the one or more length values, generating the library of polymers based on possible fragments of a parent polymer that have lengths corresponding to the one or more length values.

8. The method of claim 7, wherein the parent polymer is a protein and the possible fragments are peptides.

9. The method of claim 7, wherein the parent polymer is selected from the group consisting of deoxyribonucleic acid and ribonucleic acid and the possible fragments are nucleic acids.

10. The method of claim 1, wherein the element is chosen from the group consisting of hydrogen, carbon, nitrogen, sulfur, and phosphorous.

11. The method of claim 1, wherein the element is hydrogen, the first isotope is hydrogen-1, and the second isotope is hydrogen-2.

12. The method of claim 1, wherein the element is carbon, the first isotope is carbon-12, and the second isotope is carbon-13.

13. The method of claim 1, wherein the element is nitrogen, the first isotope is nitrogen-14, and the second isotope is nitrogen-15.

14. The method of claim 1, wherein the step of determining whether the data indicates the occurrence of the mass doublet is based on input from a user.

15. The method of claim 1, wherein the step of determining whether the data indicates the occurrence of the mass doublet comprises the computer-implemented steps of:
determining whether the data indicates that the mixture includes both the first version of the polymer and the second version of the polymer;
determining whether both a first amount of the first version and a second amount of the second version satisfy a first condition;
determining whether a ratio of the first amount to the second amount satisfies a second condition; and
determining that the data indicates the occurrence of the mass doublet when the data indicates that the mixture includes both the first version and the second version, the first amount and the second amount satisfy the first condition, and the ratio satisfies the second condition.

16. The method of claim 15, wherein the first amount and the second amount satisfy the first condition when the first amount and the second amount exceed a threshold amount.

17. The method of claim 15, wherein the ratio satisfies the second condition when the ratio is within a range based on a specified ratio and a specified error.

18. The method of claim 1, wherein the data is based on multiple scans of a chromatogram of the mixture, and wherein the step of determining whether the data indicates an occurrence of the mass doublet comprises the computer-implemented steps of:
identifying, for each scan of a plurality of the multiple scans, whether the data indicates the occurrence of the mass doublet; and
if the data for a scan indicates the occurrence of the mass doublet, then generating a first value for said scan.

19. The method of claim 18, wherein the first value is based on a first abundance of the first version and a second abundance of the second version.

20. The method of claim 18, wherein the step of determining whether the data indicates an occurrence of the mass doublet further comprises the computer-implemented steps of:
determining a number of consecutive scans of the plurality of the multiple scans for which a first value is generated; and
if the number of consecutive scans satisfies a specified condition, generating a second value.

21. The method of claim 20, wherein the step of determining whether the data indicates an occurrence of the mass doublet further comprises the computer-implemented step of:
if the data indicates the occurrence of the mass doublet, associating the second value with the polymer.

22. The method of claim 20, wherein the number of consecutive scans satisfies the specified condition when the number of consecutive scans is at least as great as a specified number of scans.

23. The method of claim 20, wherein the second value is based on the first values that are associated with the number of consecutive scans.

24. The method of claim 20, further comprising the computer-implemented step of:
determining a quantity measurement based on the second value.

25. The method of claim 1, further comprising the computer-implemented step of:
automatically determining a quantity measurement for the polymer.

26. The method of claim 25, wherein the quantity measurement is a qualitative measurement.

27. The method of claim 25, wherein the quantity measurement is a relative quantity measurement.

28. The method of claim 25, wherein the quantity measurement is an absolute quantity measurement

29. The method of claim 1, wherein the step of generating both the first mass and the second mass comprises the computer-implemented steps of:
calculating the first mass based on a first theoretical mass for the first version of the polymer; and
calculating the second mass based on a second theoretical mass for the second version of the polymer.

30. A method for identifying a polymer in a mixture, the method comprising the computer-implemented steps of:
receiving one or more length values for fragments of the polymer;
based on the one or more length values, generating a library of fragments of the polymer that have lengths corresponding to the one or more length values; and
for each fragment in the library, determining whether said fragment is present in the mixture based on a mass spectrographic analysis of the mixture.

31. The method of claim 30, wherein the one or more length values includes a minimum length.

32. The method of claim 30, wherein the one or more length values includes a maximum length.

33. The method of claim 30, wherein the one or more length values includes a minimum length and a maximum length.

34. The method of claim 30, wherein the one or more length values includes one or more ranges of lengths.

35. The method of claim 30, wherein the one or more length values includes a one or more specified length values that are received from a user.

36. The method of claim 30, wherein the step of determining includes the computer implemented steps of:
for each fragment in the library, generating both a first mass based on the fragment having a first isotope of an element and a second mass based on the fragment having a second isotope of the element;
for each fragment in the library, determining whether the mass spectrographic analysis indicates an occurrence of a mass doublet that is associated with both the first mass and the second mass.

37. A method for detecting biopolymers in a mixture that includes both natural and labeled versions of the biopolymers, the method comprising the computer-implemented steps of:
generating a library for at least one biopolymer, wherein the library includes a plurality of biopolymer fragments based on the at least one biopolymer;
determining, for each biopolymer fragment of the plurality of biopolymer fragments, both a first mass based on a natural version of the biopolymer fragment that includes a first isotope of an element and a second mass based on a labeled version of the biopolymer fragment that includes a second isotope of the element;
receiving information based on a mass spectrographic analysis of a chromatogram of the mixture, wherein the information includes data for a plurality of scans of the chromatogram;
identifying, for each scan of the plurality of scans, whether the data indicates an occurrence of one or more mass doublets, wherein each mass doublet of the one or more mass doublets is associated with both the natural version and the labeled version of a particular biopolymer fragment of the plurality of biopolymer fragments;
for each mass doublet that is identified,
generating a first score for each scan;
determining a number of consecutive scans of the plurality of scans for which the first score is generated;
if the number of consecutive scans satisfies a specified condition, generating a second score; and
associating the second score with the particular biopolymer fragment that is associated with the mass doublet.

38. The method of claim 37, further comprising the computer-implemented steps of:
receiving input that specifies a particular number of scans; and
wherein the number of consecutive scans satisfies the specified condition when the number of consecutive scans is at least as great as the particular number of scans.

39. The method of claim 37, wherein the step of identifying, for each scan of the plurality of scans, whether the data indicates the occurrence of one or more mass doublets comprises the computer-implemented steps of:
for each mass doublet of the one or more mass doublets,
determining whether the data indicates that the mixture includes both the natural version and the labeled version of the particular biopolymer fragment;
determining whether both a first abundance of the natural version and a second abundance of the labeled version exceed a threshold abundance; and
determining whether a ratio of the first abundance of the natural version to the second abundance of the labeled version is consistent with both a specified ratio and a specified error; and
identifying that the data indicates the occurrence of the mass doublet when the data indicates that the mixture includes both the natural version and the labeled version, the first abundance and the second abundance exceed the threshold abundance, and the ratio is consistent with both the specified ratio and the specified error.

40. The method of claim 39, further comprising the computer-implemented steps of:
receiving input that specifies a mass/charge accuracy associated with the mass spectrographic analysis; and
wherein the step of determining whether the data indicates that mixture includes both the natural version and the labeled version of the particular biopolymer fragment comprises the computer-implemented step of:
identifying whether a first peak occurs in the data, wherein the first peak is based on the first mass and the mass/charge accuracy;
identifying whether a second peak occurs in the data, wherein the second peak is based on the second mass and the mass/charge accuracy; and
determining that the data indicates that the mixture includes both the natural version and the labeled version of the particular biopolymer fragment when both the first peak and the second peak occur in the data.

41. The method of claim 37, wherein the step of determining both the first mass based on the natural version of the biopolymer fragment and the second mass based on the labeled version of the biopolymer fragment comprises the computer-implemented steps of:
calculating the first mass based on a first theoretical mass for the natural version of the biopolymer fragment; and
calculating the second mass based on a second theoretical mass for the labeled version of the biopolymer fragment.

42. The method of claim 41, further comprising the computer-implemented steps of:
repeating the steps of calculating the first mass and calculating the second mass for each possible charge state of the biopolymer fragment.

43. The method of claim 42, further comprising the computer-implemented step of:
receiving input that specifies one or more possible charge states of the biopolymer fragment.

44. The method of claim 37, wherein the step of generating the first score comprises the computer-implemented step of:
calculating the first score as a sum of a first average abundance that corresponds to the first mass and a second average abundance that corresponds to the second mass.

45. The method of claim 37, wherein the step of generating the second score comprises the computer-implemented step of:
calculating the second score as a summation of each first score associated with each of the number of consecutive scans.

46. The method of claim 37, further comprising the computer-implemented step of:
ranking, based on the second score for each biopolymer fragment, the one or more mass doublets that are identified.

47. The method of claim 37, further comprising the computer-implemented step of:
displaying a plot as a function of time of both a first abundance of the first mass and a second abundance of the second mass.

48. A computer-readable medium carrying one or more sequences of instructions for detecting a polymer in a mixture, wherein execution of the one or more sequences of instructions by one or more processors causes the one or more processors to perform the steps of:
generating both a first mass based on a first version of the polymer that includes a first isotope of an element and a second mass based on a second version of the polymer that includes a second isotope of the element;
receiving data based on a mass analysis of the mixture; and
determining whether the data indicates an occurrence of a mass doublet that is associated with both the first mass and the second mass.

49. The computer-readable medium of claim 48, wherein the polymer is a biopolymer.

50. The computer-readable medium of claim 49, wherein the biopolymer is comprised of one or more amino acids.

51. The computer-readable medium of claim 49, wherein the biopolymer is comprised of one or more nucleotides.

52. The computer-readable medium of claim 48, wherein the mass analysis is a mass spectrographic analysis.

53. The computer-readable medium of claim 48, wherein the polymer is a particular polymer of a plurality of polymers, and wherein the computer-readable medium further comprises instructions which, when executed by the one or more processors, cause the one or more processors to carry out the steps of:
for each polymer of the plurality of polymers, performing the steps of generating and determining.

54. The computer-readable medium of claim 53, wherein the plurality of polymers is identified in a library, and wherein the computer-readable medium further comprises instructions which, when executed by the one or more processors, cause the one or more processors to carry out the steps of:
receiving one or more length values; and
based on the one or more length values, generating the library of polymers based on possible fragments of a parent polymer that have lengths corresponding to the one or more length values.

55. The computer-readable medium of claim 54, wherein the parent polymer is a protein and the possible fragments are peptides.

56. The computer-readable medium of claim 54, wherein the parent polymer is selected from the group consisting of deoxyribonucleic acid and ribonucleic acid and the possible fragments are nucleic acids.

57. The computer-readable medium of claim 48, wherein the element is chosen from the group consisting of hydrogen, carbon, nitrogen, sulfur, and phosphorous.

58. The computer-readable medium of claim 48, wherein the element is hydrogen, the first isotope is hydrogen-1, and the second isotope is hydrogen-2.

59. The computer-readable medium of claim 48, wherein the element is carbon, the first isotope is carbon-12, and the second isotope is carbon-13.

60. The computer-readable medium of claim 48, wherein the element is nitrogen, the first isotope is nitrogen-14, and the second isotope is nitrogen-15.

61. The computer-readable medium of claim 48, wherein the step of determining whether the data indicates the occurrence of the mass doublet is based on input from a user.

62. The computer-readable medium of claim 48, wherein the instructions for determining whether the data indicates the occurrence of the mass doublet further comprise instructions which, when executed by one or more processors, cause the one or more processors to carry out the steps of:
determining whether the data indicates that the mixture includes both the first version of the polymer and the second version of the polymer;
determining whether both a first amount of the first version and a second amount of the second version satisfy a first condition;
determining whether a ratio of the first amount to the second amount satisfies a second condition; and
determining that the data indicates the occurrence of the mass doublet when the data indicates that the mixture includes both the first version and the second version, the first amount and the second amount satisfy the first condition, and the ratio satisfies the second condition.

63. The computer-readable medium of claim 62, wherein the first amount and the second amount satisfy the first condition when the first amount and the second amount exceed a threshold amount.

64. The computer-readable medium of claim 62, wherein the ratio satisfies the second condition when the ratio is within a range based on a specified ratio and a specified error.

65. The computer-readable medium of claim 48, wherein the data is based on multiple scans of a chromatogram of the mixture, and wherein the instructions for determining whether the data indicates an occurrence of the mass doublet further comprise instructions which, when executed by one or more processors, cause the one or more processors to carry out the steps of:
identifying, for each scan of a plurality of the multiple scans, whether the data indicates the occurrence of the mass doublet; and
if the data for a scan indicates the occurrence of the mass doublet, then generating a first value for said scan.

66. The computer-readable medium of claim 65, wherein the first value is based on a first abundance of the first version and a second abundance of the second version.

67. The computer-readable medium of claim 65, wherein the instructions for determining whether the data indicates an occurrence of the mass doublet further comprise instructions which, when executed by one or more processors, cause the one or more processors to carry out the steps of:
determining a number of consecutive scans of the plurality of the multiple scans for which a first value is generated; and
if the number of consecutive scans satisfies a specified condition, generating a second value.

68. The computer-readable medium of claim 67, wherein the instructions for determining whether the data indicates an occurrence of the mass doublet further comprise instructions which, when executed by one or more processors, cause the one or more processors to carry out the steps of:
if the data indicates the occurrence of the mass doublet, associating the second value with the polymer.

69. The computer-readable medium of claim 67, wherein the number of consecutive scans satisfies the specified condition when the number of consecutive scans is at least as great as a specified number of scans.

70. The computer-readable medium of claim 67, wherein the second value is based on the first values that are associated with the number of consecutive scans.

71. The computer-readable medium of claim 67, further comprising instructions which, when executed by the one or more processors, cause the one or more processors to carry out the step of:
determining a quantity measurement based on the second value.

72. The computer-readable medium of claim 48, further comprising instructions which, when executed by the one or more processors, cause the one or more processors to carry out the step of:
automatically determining a quantity measurement for the polymer.

73. The computer-readable medium of claim 72, wherein the quantity measurement is a qualitative measurement.

74. The computer-readable medium of claim 72, wherein the quantity measurement is a relative quantity measurement.

75. The computer-readable medium of claim 72, wherein the quantity measurement is an absolute quantity measurement

76. The computer-readable medium of claim 48, wherein the instructions for generating both the first mass and the second mass further comprise instructions which, when executed by one or more processors, cause the one or more processors to carry out the steps of:
calculating the first mass based on a first theoretical mass for the first version of the polymer; and
calculating the second mass based on a second theoretical mass for the second version of the polymer.

77. A computer-readable medium carrying one or more sequences of instructions for identifying a polymer in a mixture, wherein execution of the one or more sequences of instructions by one or more processors causes the one or more processors to perform the steps of:
receiving one or more length values for fragments of the polymer;
based on the one or more length values, generating a library of fragments of the polymer that have lengths corresponding to the one or more length values; and
for each fragment in the library, determining whether said fragment is present in the mixture based on a mass spectrographic analysis of the mixture.

78. The computer-readable medium of claim 77, wherein the one or more length values includes a minimum length.

79. The computer-readable medium of claim 77, wherein the one or more length values includes a maximum length.

80. The computer-readable medium of claim 77, wherein the one or more length values includes a minimum length and a maximum length.

81. The computer-readable medium of claim 77, wherein the one or more length values includes one or more ranges of lengths.

82. The computer-readable medium of claim 77, wherein the one or more length values includes a one or more specified length values that are received from a user.

83. The computer-readable medium of claim 77, wherein the instructions for determining further comprise instructions which, when executed by one or more processors, cause the one or more processors to carry out the steps of:
for each fragment in the library, generating both a first mass based on the fragment having a first isotope of an element and a second mass based on the fragment having a second isotope of the element;
for each fragment in the library, determining whether the mass spectrographic analysis indicates an occurrence of a mass doublet that is associated with both the first mass and the second mass.

84. A computer-readable medium carrying one or more sequences of instructions for detecting biopolymers in a mixture that includes both natural and labeled versions of the biopolymers, wherein execution of the one or more sequences of instructions by one or more processors causes the one or more processors to perform the steps of:
generating a library for at least one biopolymer, wherein the library includes a plurality of biopolymer fragments based on the at least one biopolymer;
determining, for each biopolymer fragment of the plurality of biopolymer fragments, both a first mass based on a natural version of the biopolymer fragment that includes a first isotope of an element and a second mass based on a labeled version of the biopolymer fragment that includes a second isotope of the element;
receiving information based on a mass spectrographic analysis of a chromatogram of the mixture, wherein the information includes data for a plurality of scans of the chromatogram;
identifying, for each scan of the plurality of scans, whether the data indicates an occurrence of one or more mass doublets, wherein each mass doublet of the one or more mass doublets is associated with both the natural version and the labeled version of a particular biopolymer fragment of the plurality of biopolymer fragments;
for each mass doublet that is identified,
generating a first score for each scan;
determining a number of consecutive scans of the plurality of scans for which the first score is generated;
if the number of consecutive scans satisfies a specified condition, generating a second score; and
associating the second score with the particular biopolymer fragment that is associated with the mass doublet.

85. The computer-readable medium of claim 84, further comprising instructions which, when executed by the one or more processors, cause the one or more processors to carry out the steps of:
receiving input that specifies a particular number of scans; and
wherein the number of consecutive scans satisfies the specified condition when the number of consecutive scans is at least as great as the particular number of scans.

86. The computer-readable medium of claim 84, wherein the instructions for identifying, for each scan of the plurality of scans, whether the data indicates the occurrence of one or more mass doublets further comprise instructions which, when executed by one or more processors, cause the one or more processors to carry out the steps of:
for each mass doublet of the one or more mass doublets,
determining whether the data indicates that the mixture includes both the natural version and the labeled version of the particular biopolymer fragment;
determining whether both a first abundance of the natural version and a second abundance of the labeled version exceed a threshold abundance; and
determining whether a ratio of the first abundance of the natural version to the second abundance of the labeled version is consistent with both a specified ratio and a specified error; and
identifying that the data indicates the occurrence of the mass doublet when the data indicates that the mixture includes both the natural version and the labeled version, the first abundance and the second abundance exceed the threshold abundance, and the ratio is consistent with both the specified ratio and the specified error.

87. The computer-readable medium of claim 86, further comprising instructions which, when executed by the one or more processors, cause the one or more processors to carry out the steps of:
receiving input that specifies a mass/charge accuracy associated with the mass spectrographic analysis; and
wherein the step of determining whether the data indicates that mixture includes both the natural version and the labeled version of the particular biopolymer fragment comprises the computer-implemented step of:
identifying whether a first peak occurs in the data, wherein the first peak is based on the first mass and the mass/charge accuracy;
identifying whether a second peak occurs in the data, wherein the second peak is based on the second mass and the mass/charge accuracy; and
determining that the data indicates that the mixture includes both the natural version and the labeled version of the particular biopolymer fragment when both the first peak and the second peak occur in the data.

88. The computer-readable medium of claim 84, wherein the instructions for determining both the first mass based on the natural version of the biopolymer fragment and the second mass based on the labeled version of the biopolymer fragment further comprise instructions which, when executed by one or more processors, cause the one or more processors to carry out the steps of:
calculating the first mass based on a first theoretical mass for the natural version of the biopolymer fragment; and
calculating the second mass based on a second theoretical mass for the labeled version of the biopolymer fragment.

89. The computer-readable medium of claim 88, further comprising instructions which, when executed by the one or more processors, cause the one or more processors to carry out the steps of:
repeating the steps of calculating the first mass and calculating the second mass for each possible charge state of the biopolymer fragment.

90. The computer-readable medium of claim 89, further comprising instructions which, when executed by the one or more processors, cause the one or more processors to carry out the steps of:
receiving input that specifies one or more possible charge states of the biopolymer fragment.

91. The computer-readable medium of claim 84, wherein the instructions for generating the first score further comprise instructions which, when executed by one or more processors, cause the one or more processors to carry out the steps of:
calculating the first score as a sum of a first average abundance that corresponds to the first mass and a second average abundance that corresponds to the second mass.

92. The computer readable medium of claim 84, wherein the instructions for generating the second score further comprise instructions which, when executed by one or more processors, cause the one or more processors to carry out the steps of:
calculating the second score as a summation of each first score associated with each of the number of consecutive scans.

93. The computer-readable medium of claim 84, further comprising instructions which, when executed by the one or more processors, cause the one or more processors to carry out the step of:
ranking, based on the second score for each biopolymer fragment, the one or more mass doublets that are identified.

94. The computer-readable medium of claim 84, further comprising instructions which, when executed by the one or more processors, cause the one or more processors to carry out the step of:
displaying a plot as a function of time of both a first abundance of the first mass and a second abundance of the second mass.
